(19) United States
(12) Patent Application Publication
(10) Pub. No.: US 2024/0173270 A1
(43) Pub. Date: May 30, 2024

(54) PHARMACEUTICAL COMPOSITION CONTAINING ETHANOL AND USE THEREOF

(71) Applicant: SUZHOU YIBEN LIFE TECHNOLOGY CO., LTD, Suzhou (CN)

(72) Inventors: Yonghua DONG, Solon, OH (US); Yuan HE, Suzhou (CN); Liangliang ZHAO, Suzhou (CN); Miaoxia LI, Suzhou (CN)

(73) Assignee: SUZHOU YIBEN LIFE TECHNOLOGY CO., LTD, Suzhou, JS (CN)

(21) Appl. No.: 18/460,656

(22) Filed: Sep. 4, 2023

Related U.S. Application Data
(63) Continuation of application No. PCT/CN2022/079386, filed on Mar. 4, 2022.

(30) Foreign Application Priority Data
Mar. 4, 2021 (CN) 202110238847.3

(51) Int. Cl. A61K 31/045; A61B 18/06; A61K 9/06; A61K 9/16; A61K 47/32; A61K 49/00; A61B 18/00

(52) U.S. Cl. CPC A61K 31/045; A61B 18/06; A61K 9/06; A61K 9/16; A61K 47/32; A61K 49/0002; A61B 2018/00416; A61B 2018/00577

(57) ABSTRACT

Disclosed are a pharmaceutical composition containing ethanol and a use thereof. The pharmaceutical composition can be used in vivo for animals, and is prepared from the following raw material: ethanol having a concentration no lower than 50% and a liquid ethanol thickener. The pharmaceutical composition containing ethanol provided in the present invention has high developability and sufficient fluidity without toxic side effect on normal tissues. The present invention does not solidify in blood and can break endothelial cells or mucous membrane superficial tissues so as to allow for permanent embolization.

### BACKGROUND

### Technical Field

The present invention relates to the technical field of interventional medicine, and particularly to a pharmaceutical composition containing ethanol and use thereof.

### Related Art

Anhydrous ethanol, due to its dehydration and erosion effect, denatures the contacted hemoglobin and directly destroys the vascular endothelial cells of abnormal blood vessels, making them lose their internal secretion function and promoting the formation of thrombosis in abnormal blood vessels, to achieve the therapeutic effect. However, direct injection of anhydrous ethanol has some problems such as easy absorption, difficulty in accurate control of the dosage, and ectopic embolism. Because of the unduly large flow rate of anhydrous ethanol, transient pulmonary hypertension and other problems will be caused.

In the existing treatment method where anhydrous ethanol is used as an embolic agent, a dual-lumen balloon or a solid embolic agent is often used, to form a cavity in a blood vessel, and then anhydrous ethanol is injected into the cavity. Although this embolization method of injecting anhydrous ethanol into the cavity is controllable, the effect is undesirable and the operation is complicated. Therefore, it is not widely used.

At present, there is another situation where a mixture of gelatin sponge and anhydrous ethanol is used to treat liver cancer complicated with moderate to severe hepatic arterioportal shunt by embolization; or the blood vessel is occluded by gelatin sponge, and then anhydrous ethanol is injected into the cavity.

It can be seen that the technical idea of embolization with anhydrous alcohol in the prior art is to form a cavity in the blood vessel, where only anhydrous alcohol is present in the cavity, to isolate the contact between blood and anhydrous alcohol.

However, when absolute ethanol itself or its mixture with gelatin sponge, is used as described above in an operation, it is invisible in the cavity under X-rays or ultrasonic waves, and an imaging agent needs to be added. This will cause the dilution of ethanol, affecting the effect of ethanol. Therefore, anhydrous ethanol will not be used, unless there is a risk of recanalization when other embolic agents are used.

Onyx, a new liquid embolic agent developed by MTI, has gradually become a widely used liquid embolic agent. However, the operation steps with Onyx include shaking the Onyx vial with a vibrator; injecting an imaging agent by using a microcatheter; rinsing the microcatheter, to wash off the imaging agent; injecting a certain amount of lowly toxic dimethyl sulfoxide (DMSO) to fill the dead space of the microcatheter; and drawing and injecting Onyx by using a special syringe. It can be seen that the operation is complicated, and adhesion to the microcatheter tends to occur, making it impossible to withdraw the microcatheter. In addition, because Onyx does not have the effect of destroying vascular endothelial cells, the potential recanalization probability is relatively high. Moreover, Onyx is expensive, which is not conducive to popularization.

### SUMMARY

In view of the above technical problems, the present invention provides a pharmaceutical composition containing ethanol and use thereof.

To achieve the above object, the following technical solutions are adopted in the present invention.

A pharmaceutical composition containing ethanol, for use in animals, comprises: ethanol with a concentration of not less than 50% and a liquid ethanol thickener.

Preferably, ethanol is added in an amount of 10 to 60 g/ml; and the liquid ethanol thickener is selected from Povidone, low-molecular glycerol, a syrup, a macromolecular hydrophilic carbohydrate polymer or hydrophilic non-carbohydrate polymer.

Preferably, the amount of the liquid ethanol thickener added is 15 to 45 g/ml.

Preferably, the pharmaceutical composition containing ethanol further comprises an imaging agent.

Preferably, the pharmaceutical composition containing ethanol further comprises a calcium acetate solution.

Preferably, the pharmaceutical composition containing ethanol further comprises an alkaline salt, selected from sodium bicarbonate, calcium carbonate, sodium acetate, sodium phosphate, sodium hypochlorite, sodium sulfite, ammonium bicarbonate, calcium hypochlorite, sodium metaaluminate or sodium carbonate. The pH value of the alkaline salt powder is 7-10.

An embolic agent or a sclerosing agent for chemical ablation is provided, which comprises the pharmaceutical composition containing ethanol.

A gas-liquid preparation comprising the pharmaceutical composition containing ethanol is provided, which comprises a plurality of sections of the pharmaceutical composition containing ethanol separated by a gas.

Use of the pharmaceutical composition containing ethanol as an embolic agent is provided.

Use of the pharmaceutical composition containing ethanol as a sclerosing agent for histochemical ablation is provided.

Use of the pharmaceutical composition containing ethanol as a sclerosing agent for mucosal surface tissues in human lumens is provided.

Use of the pharmaceutical composition containing ethanol as an anti-inflammatory agent is provided.

Use of the pharmaceutical composition containing ethanol in the preparation of drugs for treating hyperparathyroidism is provided.

Use of the pharmaceutical composition containing ethanol in the preparation of analgesics for ganglia and nerve roots is provided.

Use of the pharmaceutical composition containing ethanol in the preparation of drugs for local blood occlusion or occluding the needle passage is provided.

The pharmaceutical composition containing ethanol provided in the present invention has the following technical effects.
1. The pharmaceutical composition containing ethanol has good hydrophilicity and uniform structure, can destroy endothelial cells, and can be used as a high-performance vascular embolic agent to treat tumors by inducing ischemic necrosis of tumors and surrounding tissues.
2. The CT value, viscosity and ethanol content or concentration can be adjusted by simply adjusting the proportion of components to form a series of products, applicable to different application scenarios.
3. The blood does not need to be occluded, and the pharmaceutical composition is directly injected into the blood vessel, to improve the operation efficiency.
4. Because solid iodine and a liquid thickener are used, the ethanol concentration will not be diluted, so that the visibility in blood vessels can be realized, and the poor therapeutic effect caused by the decrease of ethanol concentration is avoided.

Therefore, the pharmaceutical composition containing ethanol provided in the present invention has high imaging ability, sufficient fluidity, and no toxic or side effects on normal tissues, will not solidify in blood, and can achieve permanent embolism by means of its destructive ability to endothelial cells.

According to the present invention, the viscosity of anhydrous ethanol is adjusted by using a thickener, to form a high-viscosity liquid or colloid with a high concentration of ethanol, allowing embolization in blood. An imaging agent is used to make the composition containing ethanol visible under X-ray or B-ultrasound, thus making it suitable for interventional surgery. By extending the action time, the concentration of ethanol is not limited to anhydrous ethanol, and lower concentration of ethanol can be used to adapt to different application scenarios (for example, to avoid vasospasm caused by excessively high concentration of ethanol. According to the present invention, anhydrous ethanol, a single concentration of embolic agent invisible under X-ray, is changed into an ethanol-containing composition with adjustable viscosity, CT value, and ethanol concentration, which is suitable for various application scenarios, and has a wider application prospect. The pharmaceutical composition containing ethanol provided in the present invention can be used as an embolic agent, which can be injected into tumor tissues, is suitable for chemical ablation, and can also be used for other pharmaceutical purposes of ethanol.

### BRIEF DESCRIPTION OF THE DRAWINGS

FIG. 1 schematically shows the effect of a fluidic embolic agent provided in the present invention;

FIG. 2 is a schematic diagram of a liquid embolic agent in Example 1 of the present invention;

FIG. 3 is a schematic diagram of a liquid embolic agent in Example 2 of the present invention;

FIG. 4 is a schematic diagram of a gelatinous embolic agent in Example 3 of the present invention;

FIG. 5 is a schematic diagram of a gelatinous embolic agent in Example 4 of the present invention;

FIG. 6 schematically shows the effect of a gelatinous embolic agent provided in the present invention;

FIG. 7 is a schematic diagram of a gelatinous embolic agent in Example 5 of the present invention;

FIG. 8 shows the test data of Examples 1 to 4 of the present invention;

FIG. 9 shows the test data of Examples 5 and 6 of the present invention; and

FIG. 10 shows the structure of an extracorporeal delivery simulating experimental device in Example 12 of the present invention.

### DETAILED DESCRIPTION

Hereinafter, the technical content of the present invention will be described in detail with reference to the attached drawings and specific embodiments.

The pharmaceutical composition containing ethanol provided in the present invention can be used in animals. It is to be noted that the animals mentioned in the present invention are mainly mammals, including but not limited to human beings and various companion animals such as cats, dogs, pigs, cattle, sheep, monkeys, and gorillas, etc., which are not elaborated herein. The pharmaceutical composition containing ethanol provided in the present invention can be used as an embolic agent, which can be injected into tumor tissues, is suitable for chemical ablation, and can also be used for other pharmaceutical purposes of ethanol. Hereinafter, an embolic agent is mainly described.

### Part I: Liquid Ethanol Embolic Agent and Preparation Method (Suitable for Microcatheter Injection Scenario)

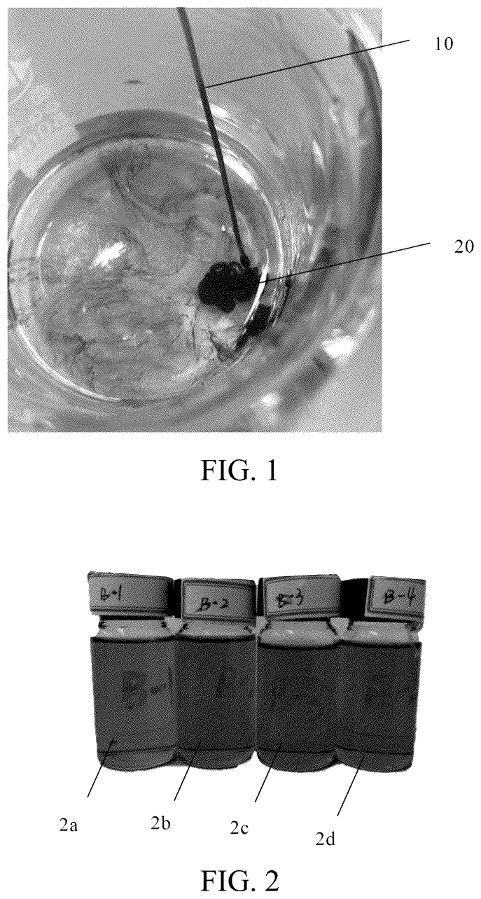

The present invention discloses a medical embolic agent containing ethanol. The embolic agent is a fluidic embolic agent 20 (as shown in FIG. 1), which comprises ethanol, an ethanol thickener and an imaging agent, and is suitable for injection with a micro-catheter 10.

Ethanol, due to its dehydration and erosion effect, denatures the contacted hemoglobin and directly destroys the vascular endothelial cells of abnormal blood vessels, thereby achieving the therapeutic purpose. Ethanol used in the present invention can be anhydrous ethanol, ethanol with a concentration of 75%, or ethanol with a concentration of 50%. However, the proportion of Povidone is correspondingly increased to increase the concentration of ethanol.

The thickener is used to adjust the viscosity of the gel, to make the embolic agent applicable to different embolization environments. In this way, the medical embolic agent is not affected by the blood flow rate, and rapid and accurate positioning can be realized. In this embodiment, description is made with Povidone as an example. A person of ordinary skill in the art should understand that other biocompatible ethanol thickeners can also be used, for example, low-molecular glycerol, a syrup, a macromolecular hydrophilic carbohydrate polymer (Arabic gum, agar, alginic acid, carboxymethyl cellulose, hydroxypropyl cellulose, maltodextrin, methyl cellulose, ethyl cellulose, pectin, sodium alginate, and xanthan gum), or a hydrophilic non-carbohydrate polymer, including gelatin, carbomer, polyoxyethylene, and polyvinyl alcohol, etc.

The imaging agent makes the pharmaceutical composition containing ethanol provided in the present invention visible in the body, which greatly improves the accurate positioning in the surgical operation process. In this embodiment, potassium iodide is taken as an example to illustrate the function of the contrast agent. A person of ordinary skill in the art should understand that other imaging agents with biocompatibility which are suitable for imaging by B-ultrasound and X-ray radiography respectively can also be used, such as an iodine preparation (e.g. iodophor), barium sulfate, and a tantalum powder, etc.

In addition, the thickener and the imaging agent can also be a compound such as Povidone iodine, which has both ethanol thickening effect and developing effect.

### Example 1

The embolic agent containing ethanol in this example includes: 200 ml of 75% ethanol, 40 g of potassium iodide and 40 to 160 g of povidone.

In this example, the iodine content is 153 mgI/ml. The preparation method of the embolic agent containing ethanol is described by way of examples in which the content of Povidone is 40 g, 80 g, 120 g, and 160 g.

Solution B-1 (Povidone 40 g): At normal temperature and under normal pressure, to a 500 ml beaker, 200 ml of 75% ethanol, and then 40 g of Povidone and 40 g of potassium iodide were added, and stirred, until the solids were dissolved into a brown liquid with high viscosity, which was used as an embolic agent. The embolic agent has a viscosity of 44 mpa·s, and a CT value of +2177 HU.

Solution B-2 (Povidone 80 g): At normal temperature and under normal pressure, to a 500 ml beaker, 200 ml of 75% ethanol, and then 80 g of Povidone and 40 g of potassium iodide were added, and stirred, until the solids were dissolved into a brown liquid with high viscosity, which was used as an embolic agent. The embolic agent has a viscosity of 256 mpa·s, and a CT value of +1846 HU.

Solution B-3 (Povidone 120 g): At normal temperature and under normal pressure, to a 500 ml beaker, 200 ml of 75% ethanol, and then 120 g of Povidone and 40 g of potassium iodide were added, and stirred, until the solids were dissolved into a brown liquid with high viscosity, which was used as an embolic agent. The embolic agent has a viscosity of 956 mpa·s, and a CT value of +1727 HU.

Solution B-4 (Povidone 160 g): At normal temperature and under normal pressure, to a 500 ml beaker, 200 ml of 75% ethanol, and then 160 g of Povidone and 40 g of potassium iodide were added, and stirred until the solids were dissolved into a brown liquid with high viscosity, which was used as an embolic agent. The embolic agent has a viscosity of 3116 mpa·s, and a CT value of +1779 HU.

As shown in FIG. 2, 2a is the embolic agent of Solution B-1, 2b is the embolic agent of Solution B-2, 2c is the embolic agent of Solution B-3, and 2d is the embolic agent of Solution B-4.

According to the experiment of this example, embolic agents with a viscosity of less than 956 mpa·s can be injected by micro-catheter.

### Example 2

The embolic agent in this example includes: 200 ml of anhydrous ethanol and 37.4 to 187 g of Povidone. Hereinafter, the preparation method of the embolic agent is described by way of examples in which the iodine content is 20 mgI/ml, 60 mgI/ml, and 100 mgI/ml respectively.

Solution A-1 (iodine content 20 mg I/ml): At normal temperature and under normal pressure, to a 500 ml beaker, 200 ml of anhydrous ethanol, and then 37.4 g of Povidone were added, and stirred until the solid was dissolved into a brown liquid with high viscosity, which was used as an embolic agent. The embolic agent has a viscosity of 20 mpa·s, and a CT value of +405 HU.

Solution A-2 (iodine content 60 mgI/ml): At normal temperature and under normal pressure, to a 500 ml beaker, 200 ml of anhydrous ethanol, and then 112 g of Povidone were added, and stirred until the solid was dissolved into a brown liquid with high viscosity, which was used as an embolic agent. The embolic agent has a viscosity of 794 mpa·s, and a CT value of +1129 HU.

Solution A-3 (iodine content 100 mgI/ml): At normal temperature and under normal pressure, to a 500 ml beaker, 200 ml of anhydrous ethanol, and then 187 g of Povidone were added, and stirred until the solid was dissolved into a brown liquid with high viscosity, which was used as an embolic agent. The embolic agent has a viscosity of 10981 mpa·s, and a CT value of +1580 HU.

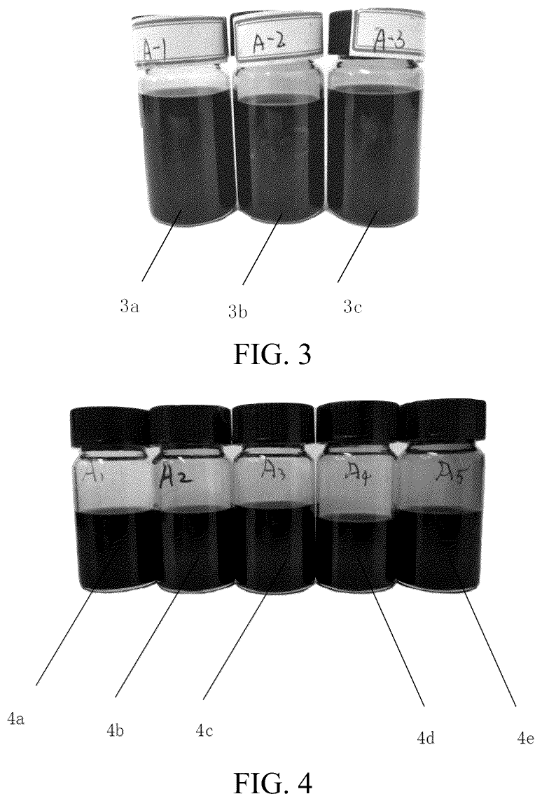

As shown in FIG. 3, 3a is the embolic agent of Solution A-1, 3b is the embolic agent of Solution A-2, and 3c is the embolic agent of Solution A-3.

According to the experiment of this example, embolic agents with a viscosity of less than 2000 mpa·s can be injected into the blood vessel by a micro-catheter.

During use, a common injection needle is directly used (no special needle like Onyx is needed), to draw the pharmaceutical composition containing ethanol provided in the present invention out of a vial, and inject it into the body through a microcatheter. In Onyx series products, a tantalum powder of 10 microns or less, DMSO and EVOH is prepared into a suspension, which needs to be shaken continuously for more than 20 min before injection. The liquid embolic agent provided in the present invention does not require this operation.

The injection speed of the liquid embolic agent containing ethanol provided in the present invention can exceed 0.16 ml/min. The liquid embolic agent, due to its liquid or gel state, does not take time to solidify from outside to inside as Onyx does, and can avoid vasospasm caused by DMSO. Compared with Onyx18, Onyx20 or Onyx34 series products, the embolic agent provided in the present invention has higher viscosity, can be quickly embolized in target blood vessels, and filled in abnormal blood vessels with pathological changes. Moreover, ethanol has a destructive effect on vascular endothelial cells, and can avoid the formation of new blood vessels, thus preventing vascular recanalization. The injection speed is high (up to 0.2 ml/min).

With the aid of medical imaging systems such as B ultrasound, CT, X-ray, and DSA, the recanalization of blood vessels and the formation of new collateral vessels can be evaluated by imaging examination. The high-viscosity liquid embolic agent enables high-definition imaging, has good dispersibility, is uneasy to be washed away by blood flow, can fully occlude the whole blood vessel, has good controllability and no obvious toxic and side effects, will not cause improper embolization, and has standard dosage.

### Example 3

The embolic agent in this example includes: 10 ml of anhydrous ethanol and 1.87 to 9.35 g of Povidone.

The preparation method of the embolic agent is described by way of examples in which the iodine content is 20 mgI/ml, 40 mgI/ml, 60 mgI/ml, 80 mgI/ml, and 100 mgI/ml respectively.

Solution A1 (iodine content 20 mgI/ml): At normal temperature and under normal pressure, to a 50 ml beaker, 10 ml of anhydrous ethanol, and then 1.87 g of Povidone were added, and stirred until the solid was dissolved into a brown liquid with high viscosity, which was used as an embolic agent. The embolic agent has a CT value of +378 HU.

Solution A2 (iodine content 40 mgI/ml): At normal temperature and under normal pressure, to a 50 ml beaker, 10 ml of anhydrous ethanol, and then 3.73 g of Povidone were added, and stirred until the solid was dissolved into a brown liquid with high viscosity, which was used as an embolic agent. The embolic agent has a CT value of +753 HU.

Solution A3 (iodine content 60 mgI/ml): At normal temperature and under normal pressure, to a 50 ml beaker, 10 ml of anhydrous ethanol, and then 5.6 g of Povidone were added, and stirred until the solid was dissolved into a brown liquid with high viscosity, which was used as an embolic agent. The embolic agent has a CT value of +993 HU. Tests show that from the time when the microcatheter enters the blood (physiological saline), the embolic agent is diluted or decomposed into a biocompatible water-soluble substance without toxicity and side effects after about 20 seconds, and no water-insoluble substance is left in the blood.

Solution A4 (iodine content 80 mgI/ml): At normal temperature and under normal pressure, to a 50 ml beaker, 10 ml of anhydrous ethanol, and then 7.47 g of Povidone were added, and stirred until the solid was dissolved into a brown liquid with high viscosity, which was used as an embolic agent. The embolic agent has a CT value of +1597 HU.

Solution A5 (iodine content 100 mgI/ml): At normal temperature and under normal pressure, to a 50 ml beaker, 10 ml of anhydrous ethanol, and then 9.35 g of Povidone were added, and stirred until the solid was dissolved into a brown liquid with high viscosity, which was used as an embolic agent. The embolic agent has a CT value of +1650 HU.

Solution A6: 50 ml of pure water and 50 g of Povidone iodine were added into 50 ml absolute ethanol, to obtain an embolic agent, which was injected in physiological saline to form a string-like liquid (as shown in FIG. 1). It can be seen that even if a low concentration of ethanol is used, an appropriate amount of povidone iodine can be used to make it a liquid embolic agent with high viscosity, so the contact time between embolic agent and endothelial cells can be prolonged.

Tests show that the embolic agents prepared in Solutions A1 to A6 can be diluted or decomposed into a biocompatible water-soluble substance without toxicity and side effects after about 20 seconds in physiological saline. In other words, no matter what the concentration of ethanol is, the time of action of the embolic agent prepared according to the preparation method provided in the present invention in blood (physiological saline) can be controlled (the length of time can be adjusted by adjusting the proportion of povidone), unlike anhydrous ethanol that only has a very short time of action in physiological saline. It can be understood that in practical use, because the flow rate of blood will shorten the time of action of liquid embolic agents, an embolic agent with high viscosity (regardless of the concentration of ethanol) can be injected into blood vessels with fast blood flow rate, to increase the time of action. Further, because of the prolonged time of action, even if an embolic agent with low ethanol concentration provided in the present invention is used, the same or similar therapeutic effect as the anhydrous ethanol embolic agent can be achieved, and vasospasm can be relieved.

As shown in FIG. 4, 4a is the embolic agent of Solution A1, 4b is the embolic agent of Solution A2, 4c is the embolic agent of Solution A3, 4d is the embolic agent of Solution A4, and 4e is the embolic agent of Solution A5. The embolic agent obtained in this example has good fluidity, and does not form a gel, but a liquid with higher viscosity (the viscosity is lower than that in Example 2, but higher than that of anhydrous ethanol) after being injected into blood vessels, and is suitable for being injected into blood vessels or tumor tissues by a microcatheter.

### Example 4

The embolic agent in this example includes: 10 ml of 75% ethanol, 5 g of Povidone and 0.2 to 2.6 g of potassium iodide.

The preparation method of the embolic agent is described by way of examples in which the iodine content is 15.3 mgI/ml, 50 mgI/ml, 100 mgI/ml, 153 mgI/ml, and 200 mgI/ml respectively.

Solution B1 (iodine content 15.3 mgI/ml): to a 50 ml beaker, 10 ml of 75% ethanol, and then 5 g of Povidone and 0.2 g of potassium iodide were added, and stirred until the solids were dissolved into a brown liquid with high viscosity, which was used as an embolic agent. The embolic agent has a CT value of +240 HU.

Solution B2 (iodine content 50 mgI/ml): to a 50 ml beaker, 10 ml of 75% ethanol, and then 5 g of Povidone and 0.65 g of potassium iodide were added, and stirred until the solids were dissolved into a brown liquid with high viscosity, which was used as an embolic agent. The embolic agent has a CT value of +617 HU.

Solution B3 (iodine content 100 mgI/ml): to a 50 ml beaker, 10 ml of 75% ethanol, and then 5 g of Povidone and 1.3 g of potassium iodide were added, and stirred until the solids were dissolved into a brown liquid with high viscosity, which was used as an embolic agent. The embolic agent has a CT value of +1225 HU.

Solution B4 (iodine content 153 mg/ml): to a 50 ml beaker, 10 ml of 75% ethanol, and then 5 g of Povidone and 2 g of potassium iodide were added, and stirred until the solids were dissolved into a brown liquid with high viscosity, which was used as an embolic agent. The embolic agent has a CT value of +1934 HU.

Solution B5 (iodine content 200 mgI/ml): to a 50 ml beaker, 10 ml of 75% ethanol, and then 5 g of Povidone and 2.6 g of potassium iodide were added, and stirred until the solids were dissolved into a brown liquid with high viscosity, which was used as an embolic agent. The embolic agent has a CT value of +2492 HU.

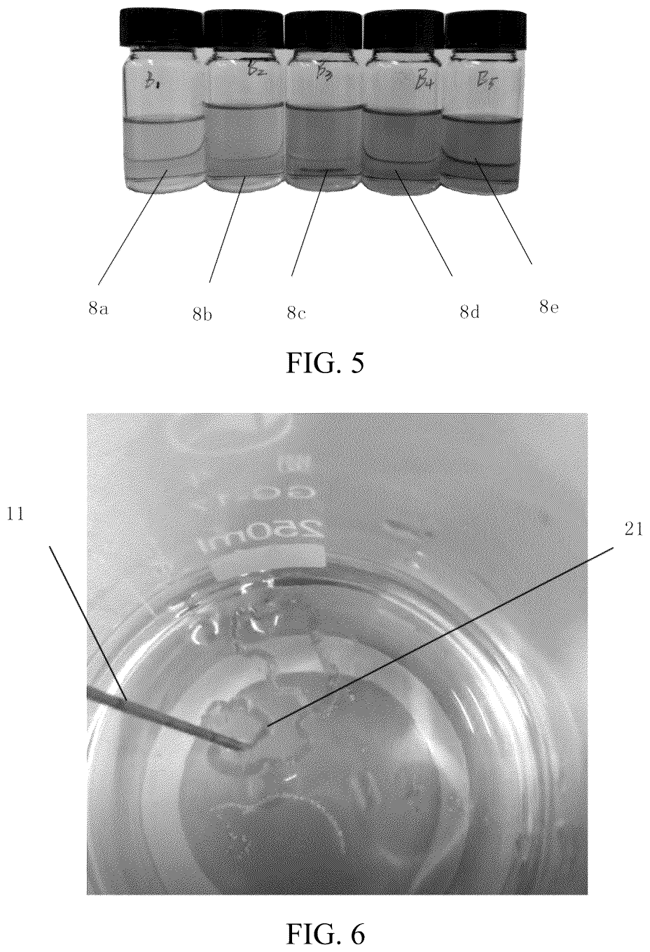

As shown in FIG. 5, 8a is the embolic agent of Solution B1, 8b is the embolic agent of Solution B2, 8c is the embolic agent of Solution B3, 8d is the embolic agent of Solution B4, and 8e is the embolic agent of Solution B5.

The embolic agent obtained in this example has good fluidity, and is particularly suitable for injection into blood vessels by using a conventional microcatheter. The test data is shown in Table 1 below:

TABLE 1
Viscosity and injection grading of embolic agents prepared
with povidone iodine and absolute ethanol

| | Mass-volume | | | | | |
|---|---|---|---|---|---|---|
| | ratio of | Anhydrous | | | Iodine | |
| | Povidone | ethanol | Povidone | Viscosity | content, | Injection |
| No. | iodine (g/ml) | (ml) | iodine (g) | (mpa · s) | mgI/ml | grading |
| 1 | 10% | 400 | 40 | 8.6 | 10.7 | Easy |
| 2 | 20% | 400 | 80 | 27.6 | 21.4 | Easy |
| 3 | 30% | 400 | 120 | 88 | 32.1 | Easy |
| 4 | 40% | 400 | 160 | 264.5 | 42.8 | Easy |
| 5 | 50% | 400 | 200 | 753 | 53.5 | Easy |
| 6 | 55% | 400 | 220 | 1205 | 58.85 | Easy |
| 7 | 60% | 400 | 240 | 2000 | 64.2 | Injection |
| | | | | | | with large |
| | | | | | | force |
| 8 | 65% | 400 | 260 | 3400 | 69.55 | Hard |

As can be seen from Table 1 above, when the mass-volume ratio of povidone iodine exceeds 60% g/ml and the viscosity reaches 2000 mpa·s, the embolic agent prepared is not suitable for surgical operation. In a preferred solution, when the mass-volume ratio of Povidone iodine is less than 55% g/ml, that is, the viscosity is less than 1200 mpa·s, the embolic agent prepared can be used as an embolic agent or sclerosing agent in surgery.

As can be seen from the following Table 2, when the mass-volume ratio of Povidone iodine exceeds 65% g/ml and the viscosity exceeds 2000 mpa·s, the embolic agent prepared is not suitable for surgical operation. In a preferred solution, when the mass-volume ratio of Povidone iodine is less than 60% g/ml, that is, the viscosity is less than 1470 mpa·s, the embolic agent prepared can be used as an embolic agent or sclerosing agent in surgery.

TABLE 2
Viscosity and injection grading of embolic agents
prepared with povidone and absolute ethanol

| | Mass-volume | | | | |
|---|---|---|---|---|---|
| | ratio of | Anhydrous | | | |
| | Povidone | ethanol | Povidone | Viscosity | Injection |
| No. | iodine (g/ml) | (ml) | iodine (g) | (mpa · s) | grading |
| 1 | 10% | 400 | 40 | 10.6 | Easy |
| 2 | 20% | 400 | 80 | 31 | Easy |
| 3 | 30% | 400 | 120 | 90.6 | Easy |
| 4 | 40% | 400 | 160 | 236 | Easy |
| 5 | 50% | 400 | 200 | 589 | Easy |
| 6 | 55% | 400 | 220 | 905 | Easy |
| 7 | 60% | 400 | 240 | 1470 | Relatively easy |
| 8 | 65% | 400 | 260 | 2650 | Hardly |
| | | | | | injectable |

As can be seen from Tables 1 and 2, when the viscosity exceeds 2000 mpa·s, a greater injection force is needed. Therefore, the pharmaceutical composition containing ethanol and has a viscosity of 2000 mpa·s or less provided in the present invention is recommended to be used as an embolic agent (injected by a microcatheter). It can be understood by those skilled in the art that if the injection is performed with a puncture needle instead, the viscosity is not limited. In the case of injection by a puncture needle, the injection is feasible even if the mass-volume ratio of Povidone exceeds 85% (but whether it is an effective drug needs to be determined according to the actual application scenario).

### Part II: Gelatinous Ethanol Embolic Agent and Preparation Method

The pharmaceutical composition containing ethanol in this example can also be a paste or gelatinous composition 21 (as shown in FIG. 6), which is suitable for injection into blood vessels or tissues (such as tumor tissues) by a puncture needle 11.

The medical embolic agent containing ethanol in this example comprises ethanol, calcium acetate, an ethanol thickener and an imaging agent.

Ethanol, due to its dehydration and erosion effect, denatures the contacted hemoglobin and directly destroys the vascular endothelial cells of abnormal blood vessels, thereby achieving the therapeutic purpose.

Calcium acetate (CH₃COO—Ca—OOCCH₃) is hardly soluble in ethanol. However, calcium acetate carries the organic group CH₃COO—, which attracts the organic group CH₃CH₂— in ethanol. A small amount of calcium acetate can be evenly dispersed in ethanol. Therefore, when calcium acetate is in contact with ethanol, it will precipitate into a gel. For example, when 5% calcium acetate (3 g calcium acetate+57 ml water) is added to 40 g of Povidone and 100 ml of anhydrous ethanol, a gel is formed, but it is soft (lowly viscous).

The viscosity of the gel can be adjusted with an ethanol thickener, to make the embolic agent containing ethanol applicable to different embolization environments. In this way, the embolic agent is not affected by the blood flow rate, and rapid and accurate positioning can be realized. In this embodiment, description is made with Povidone as an example. A person of ordinary skill in the art should understand that other biocompatible ethanol thickeners can also be used, for example, low-molecular glycerol, a syrup, a macromolecular hydrophilic carbohydrate polymer (Arabic gum, agar, alginic acid, carboxymethyl cellulose, hydroxypropyl cellulose, maltodextrin, methyl cellulose, ethyl cellulose, pectin, sodium alginate, xanthan gum), or a hydrophilic non-carbohydrate polymer, including gelatin, carbomer, polyoxyethylene, and polyvinyl alcohol, etc.

According to the embolic agent containing ethanol provided in the present invention, by controlling the dosage of the ethanol thickener (for example, Povidone), the viscosity of the embolic agent is adjusted, so that the embolic agent will not adhere to the wall. By accurately controlling the dosage, the therapeutic effect is improved.

The imaging agent makes the gel embolic agent visible in blood vessels when imagined. In this example, potassium iodide is taken as an example to illustrate the function of the contrast agent. However, a person of ordinary skill in the art should understand that other imaging agents with biocompatibility which are suitable for imaging by B-ultrasound and X-ray radiography respectively can also be used, such as an iodine preparation (e.g. lipiodol, Ioversol, and lodixanol), barium sulfate, and a tantalum powder, etc. The ethanol thickener and the imaging agent can be combined, for example, Povidone iodine.

### Example 5

The embolic agent in this example includes: 200 ml of anhydrous ethanol, 37.4 to 187 g of Povidone iodine, 10 ml of a calcium acetate solution (calcium acetate:water=35:100). The iodine content in Povidone iodine is 20 mgI/ml to 100 mgI/ml.

Hereinafter, the preparation method of the embolic agent in this example is described.

Solution A-1/C (iodine content 20 mgI/ml): At normal temperature and under normal pressure, to a 500 ml beaker, 200 ml of anhydrous ethanol and 37.4 g of Povidone were added, and stirred to dissolve the solid into a uniform solution. 10 ml of a calcium acetate aqueous solution was slowly added with stirring, until the solution became a brown gel, which was used as an embolic agent. The embolic agent of this Solution has a CT value of +440 HU (Hounsfield). Tests show that the gelatinous embolic agent prepared according to Solution A-1/C will be automatically decomposed or hydrolyzed into a biocompatible water-soluble substance without toxicity or side effects after about 40 seconds from the time when it is pushed out of the needle passage and enters the blood, and no water-insoluble substance is left in the blood vessel.

Solution A-2/C (iodine content 60 mgI/ml): At normal temperature and under normal pressure, to a 500 ml beaker, 200 ml of anhydrous ethanol and 112 g of Povidone were added, and stirred to dissolve the solid into a uniform solution. 10 ml of a calcium acetate aqueous solution was slowly added with stirring, until the solution became a brown gel, which was used as an embolic agent. The embolic agent has a CT value of +1129 HU.

Solution A-3/C (iodine content 100 mg/ml): At normal temperature and under normal pressure, to a 500 ml beaker, 200 ml of anhydrous ethanol, and then 187 g of Povidone were added, and stirred to dissolve the solid into a uniform solution. 10 ml of a calcium acetate aqueous solution was slowly added with stirring, until the solution became a brown gel, which was used as an embolic agent. The embolic agent has a CT value of +1580 HU.

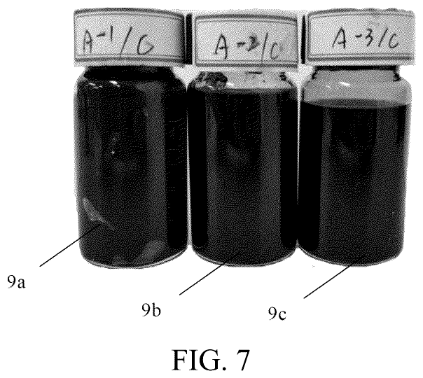

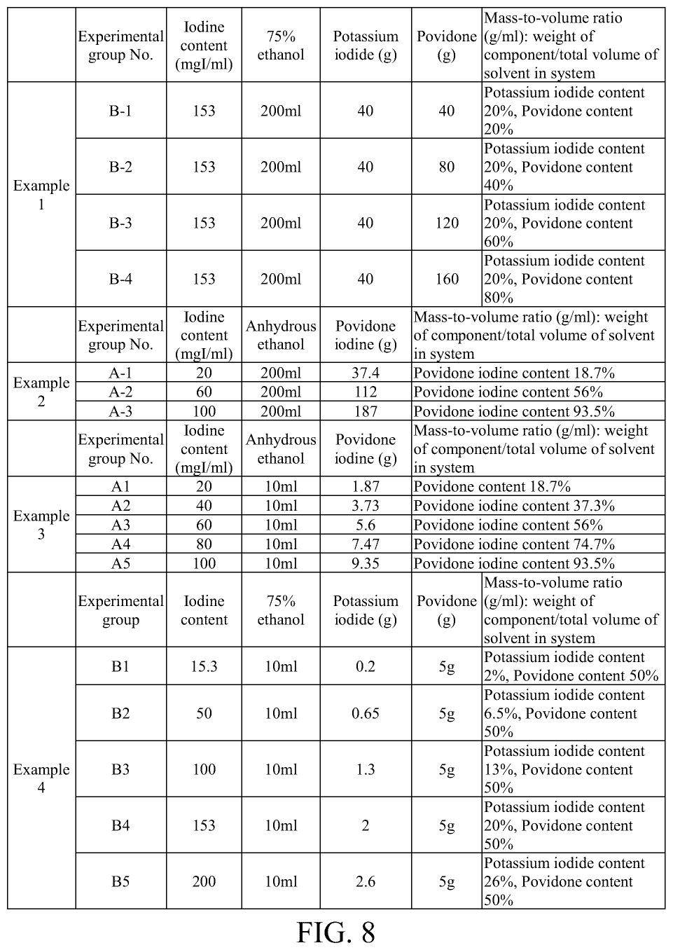

As shown in FIG. 7, 9a is the embolic agent of Solution A-1/C, 9b is the embolic agent of Solution A-2/C, and 9c is the embolic agent of Solution A-3/C.

### Example 6

The embolic agent in this example includes: 200 ml of 75% ethanol, 40 g of potassium iodide, 40 to 160 g of Povidone, and 10 ml of a calcium acetate solution (calcium acetate:water=35:100). The iodine content is 153 mgI/ml.

Hereinafter, the preparation method of the embolic agent in this example is described.

Solution B-1/C (iodine content 153 mgI/ml): At normal temperature and under normal pressure, to a 500 ml beaker, 40 g of Povidone, 40 g of potassium iodide, and 3.5 g of calcium acetate were dissolved in 50 ml of pure water, and stirred to dissolve the solids into a uniform solution. 150 ml of anhydrous ethanol was slowly added with stirring, until the solution became a brown gel, which was used as an embolic agent. The embolic agent of this Solution has a CT value of +2206 HU.

### Example 7

The embolic agent in this example includes: 200 ml of 75% ethanol, 40 g of potassium iodide, 160 g of Povidone, and 10 ml of a calcium acetate solution (calcium acetate:water=35:100). The iodine content is 153 mgI/ml.

Hereinafter, the preparation method of the embolic agent in this example is described.

Solution B-4/C (iodine content 153 mgI/ml): At normal temperature and under normal pressure, to a 500 ml beaker, 160 g of Povidone, 40 g of potassium iodide, and 3.5 g of calcium acetate were dissolved in 50 ml of pure water, and stirred to dissolve the solids into a uniform solution. 150 ml of anhydrous ethanol was slowly added with stirring, until the solution became a brown gel, which was used as an embolic agent. The embolic agent of this Solution has a CT value of +1949 HU.

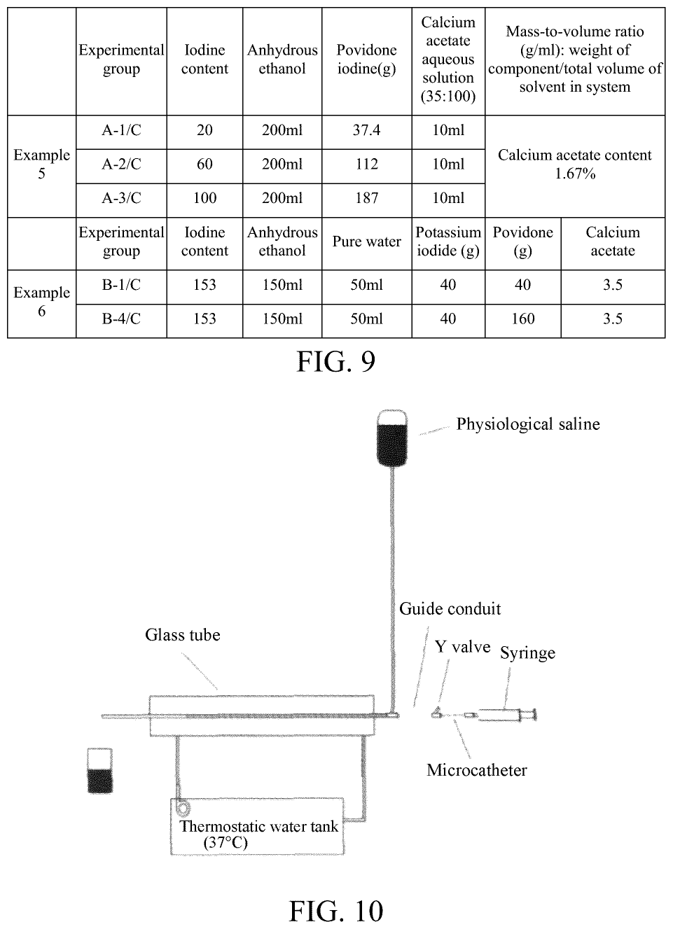

When the composition of each of the above examples is injected into blood vessels, a puncture needle is used to directly inject it into the blood vessels or tissues in the target area. Some test data are shown in FIG. 9.

A large number of theoretical tests show that the medical embolic agent provided in the present invention has high vascular compliance, good hydrophilicity, uniform structure, and good plasticity, and can be densely and reliably adhered to the blood vessel after injection, for use as a high-performance vascular embolic agent to block blood vessels for treatment.

### Part III: Microsphere Solution

Microspheres can be added to the liquid or gel embolic agent provided in the present invention, so as to improve the drug carrying capacity. Studies show that both suspensions of hydrogel microspheres and granular hydrogels can be used for minimally invasive delivery of biological agents, that is, the hydrogel microspheres can be dissolved in a solvent and injected into the patients' tissues, or various hydrogel microspheres are mixed together to obtain a multi-functional sclerosing agent. For example, two different drugs can be encapsulated in different particles.

### Example 8

The preparation method of a fluidic embolic agent with drug carrying microspheres is described below. The embolic agent in this example includes: 10 g of 75% ethanol, 5 g of Povidone, 2 g of potassium iodide, and an appropriate drug carrying microspheres. The drug on the drug carrying microspheres should not react with the above components, meet the pharmaceutical requirements, and be provided in an amount that is controlled within a range meeting the hemodynamic requirements, as long as it can be smoothly ejected from the catheter.

S11: At normal temperature and under normal pressure, 2 g of potassium iodide was added to 10 g of anhydrous ethanol, to obtain a mixed ethanol solution.

S12: 2 ml of a saturated calcium acetate solution was prepared at normal temperature and, 5 g of Povidone was dissolved, to prepare a mixed calcium acetate solution.

S13: The mixed ethanol solution was fed to a 50 ml beaker, to which the mixed calcium acetate solution and a proper amount of micron-scale drug carrying microspheres, slowly added with stirring, until the solution became a brown gel, which was used as an embolic agent.

### Example 9

The embolic agent in this example includes: 10 g of anhydrous ethanol, 5.6 g of Povidone, and 0.3 g of drug carrying microspheres.

The preparation method of the embolic agent was as follows. To a 50 ml beaker, 10 g of anhydrous ethanol, and then 5.6 g of Povidone iodine and 0.3 g of drug carrying microspheres having a micro-scale particle size of 100 to 2000 μm were added, and stirred until the solids were dissolved into a brown liquid with high viscosity, which was used as an embolic agent.

### Example 10

The embolic agent in this example includes: 50 ml of ethanol, 50 ml of water, 50 g of Povidone iodine and 4.5 g of drug carrying microspheres. During injection into physiological saline, the drug carrying microspheres are evenly dispersed in the embolic agent and have a long time of action because the concentration of ethanol does not affect the diffusion time (action time).

### Part IV: Tumor Sclerosing Agent Achieving Acid-Base Balance

### Example 11

It is well known that glycolysis of tumor cells under anaerobic conditions will produce a large amount of the intermediate product: lactic acid. In addition, the metabolism in tumors is mainly anaerobic glycolysis, which consumes more “energy”, and needs more energy than normal tissue, and is quite sensitive to ischemia. Tumor vascular endothelial cells proliferate 20 to 2000 times faster than normal tissues, and are also intolerant to ischemia and hypoxia. Therefore, the composition containing ethanol provided in the present invention is used as an embolic agent, in which povidone is used to increase the viscosity and ethanol concentration of the composition, and a small amount of a base (such as sodium bicarbonate and calcium carbonate) was added to adjust the pH to 8 to 9, to make the tumor cells ischemic, destroy the tumor vascular endothelial cells, neutralize the lactic acid, and realize an acid-base balance environment in tumor tissues. Therefore, use of the composition containing ethanol provided in the present invention as an embolic agent or a sclerosing agent can further improve the therapeutic effect on tumors.

| | Specification | Dosage | Amount of | Concentration |
|---|---|---|---|---|
| | of ethanol | of ethanol | sodium | of sodium |
| No. | embolic agent | embolic agent | bicarbonate | bicarbonate |
| 1 | Anhydrous | 30 μl | 5 μl | 0.02 mol/L |
| | ethanol | | | |
| 2 | 15% PVP | 30 μl | 5 μl | 0.05 mol/L |
| 3 | 20% PVP | 30 μl | 5 μl | 0.1 mol/L |
| 4 | 25% PVP | 30 μl | 5 μl | 0.5 mol/L |

### Example 12

FIG. 10 shows the structure of an extracorporeal delivery simulating experimental device in Example 12 of the present invention. The device includes a thermostatic water tank, a glass tube, a physiological saline bottle, a guide conduit, a Y valve, a microcatheter and a syringe. The thermostatic water tank is located below the glass tube, and the glass tube which is connected with the physiological saline bottle through a conduit. The conduit is connected to the guide conduit at a middle position, the guide conduit is connected to the microcatheter through the Y valve, and the microcatheter is connected to the syringe.

In this embodiment, CO₂ is taken as an example to explain the gas-liquid preparation of the pharmaceutical composition containing ethanol, but it may be replaced by oxygen or air. In this embodiment, the gas-liquid preparation of the pharmaceutical composition containing ethanol is a serial pharmaceutical preparation formed by separating a plurality of sections of the pharmaceutical composition containing ethanol by a gas, and used for the treatment of humans. For a detailed description of the gas-liquid preparation, please refer to Chinese Patent Application No. 2020213527786, entitled “Gas-liquid conveying joint, gas-liquid conveying joint pair and gas-liquid delivery device” and U.S. Pat. No. 10,695,018 entitled “Train-like pharmaceutical configuration, apparatus for preparation and storage device thereof”.

### 1. The Test Instruments/Drugs are Shown in Table 3 Below:

TABLE 3

| No. | Instrument/drug | Remark |
|---|---|---|
| 1 | Anhydrous ethanol | |
| 2 | Povidone K30 | PVPK30 |
| 3 | Physiological saline | 0.90% |
| 4 | Rotational viscometer | NDJ-9s digital viscometer produced |
| | | by Shanghai Fang Rui Instrument |
| | | Co., Ltd. |
| 5 | Rotor (0/1/2/3/4) | Routine device |
| 6 | Microcatheter, | Diameter 5F |
| 7 | Peristaltic pump | NKCP-S10 produced byKAMOER |
| 8 | Injection device | Routine device |

### 2. Preparation of Test Samples

The dynamic viscosity values of ethanol embolic agents with different specifications are shown in Table 4 below:

TABLE 4

| | Mass-volume ratio | | | | |
|---|---|---|---|---|---|
| | of Povidone to | | | | |
| | anhydrous ethanol | | | | |
| | (g/ml) | | | | |
| | (Specification | Anhydrous | | | Specification |
| | of ethanol | ethanol | Povidone | Viscosity | of corresponding |
| No. | embolic agent) | (ml) | (g) | (mpa · s) | Onyx product |
| 1 | 15% | 400 | 60 | 21.1 | onyx20 |
| 2 | 20% | 400 | 80 | 32.6 | onyx34 |
| 3 | 25% | 400 | 100 | 50.5 | |
| 4 | 30% | 400 | 120 | 74 | |
| 5 | 35% | 400 | 140 | 114.6 | |
| 6 | 40% | 400 | 160 | 171 | |
| 7 | 45% | 400 | 180 | 270.5 | |

### 3. Extracorporeal Delivery Simulating Experiment

A gas-liquid injection device is connected for injection, and the alcohol injection speed is recommended to be 0.1 to 1 ml/s. As a reference, the injection speed of Onyx products is recommended to be 0.1 to 0.3 ml/min.

In this experiment, the injection speed of ethanol embolic agent was controlled by controlling the inner diameter of the conveying tube to 3 mm, and the speed of the peristaltic pump to 1 ml/s. The delivery status of ethanol embolic agents with different viscosities at different speeds were observed. The results are shown in Table 5 below.

TABLE 5

| | | | | |
|---|---|---|---|---|
| Speed of pump ml/s | 1 | 0.8 | 0.5 | 0.3 |
| Diameter cm | 0.3 | 0.3 | 0.3 | 0.3 |
| Radius cm | 0.15 | 0.15 | 0.15 | 0.15 |
| Area cm | 0.07 | 0.07 | 0.07 | 0.07 |
| Flow rate cm/s | 14.15 | 11.32 | 7.08 | 4.25 |

### 4. Experimental Design

### 4.1 Determination of Viscosity Deviation

For each group (15%, 20%, 25%, 30%, 35%, 40%; and 45%), 5 samples were set, and the viscosity was measured to determine the range of the viscosity deviation.

### 4.2 Extracorporeal Delivery Experiment

Each concentration of ethanol embolic agent was tested at a speed of 1 ml/s of the delivery pump, and the injection speed was tentatively set at 0.1 ml/min, 0.1 ml/s, and 1 ml/s. That is, 3 tests were set for each concentration. The delivery of the ethanol embolic agent was observed.

Various concentrations of ethanol embolic agent, an imaging agent, and carbon dioxide were prepared into a new dosage form with different proportions, which was then subjected to injection tests at different speeds. The flow state of the new dosage form in an extracorporeal flowing conduit in the extracorporeal delivery simulation was observed, to ensure the intermittent delivery of the three.

By using anhydrous ethanol, 15% PVP ethanol embolic agent, 20% PVP ethanol embolic agent, and 25% PVP ethanol embolic agent shown in Table 4, the ethanol embolic agents (in the form of gas-liquid preparations) prepared are shown in Table 6 below:

TABLE 6

| | Specification | Volume of | Volume of | | |
|---|---|---|---|---|---|
| | of ethanol | ethanol | imaging | Volume | Injection |
| No. | embolic agent | embolic agent | agent | of CO₂ | speed |
| 1 | Anhydrous | 30 μl | 5 μl | 15 μl | 50 μl/s |
| | ethanol | | | | |
| 2 | 15% PVP | 30 μl | 5 μl | 40 μl | 10 μl/s |
| 3 | 20% PVP | 30 μl | 5 μl | 40 μl | 5 μl/s |
| 4 | 25% PVP | 30 μl | 5 μl | 60 μl | 5 μl/s |

Note: If mixing occurs during injection, the volume of CO₂ is adjusted and the injection speed is reduced.

During injection of 25% ethanol embolic agent (with a viscosity of 50.5 mpa·s) at a delivery speed of 1 ml/s (14 cm/s) and an injection speed of 0.1 ml/s or 0.05 ml/s, the push force needed is 1 Mpa, the push rod of the injection device is broken, and the syringe was deformed. A 10 ml syringe was changed, the push rod was improved, and then the test was carried out. The viscosity data is shown in Table 7 below:

TABLE 7

| | Mass- | | | | | |
|---|---|---|---|---|---|---|
| | volume ratio | Viscosity (mpa · s) | | | | |
| No. | g/ml | 1# | 2# | 3# | 4# | 5# |
| 1 | 5% PVP | 4.22 | 4.32 | 4.42 | 4.48 | 4.74 |
| 2 | 10% PVP | 8.05 | 7.84 | 8.1 | 7.66 | 7.68 |
| 3 | 15% PVP | 14.24 | 15.08 | 14.04 | 13.78 | 14.2 |
| 4 | 20% PVP | 36.4 | 33.3 | 34.4 | 35.2 | 32.9 |
| 5 | 25% PVP | 43.6 | 46.1 | 47.5 | 45.7 | 49.5 |
| 6 | 30% PVP | 64 | 69.5 | 65.3 | 65 | 67 |
| 7 | 35% PVP | 97 | 101.6 | 100.8 | 98.6 | 86.8 |

The viscosity depends on the K number of Povidone. If Povidone K30 is used, the viscosity is in the range of 27 to 32.

The injection recording in the extracorporeal delivery simulating experimental is shown in Table 8:

TABLE 8

| | | | | | |
|---|---|---|---|---|---|
| | Embolic agent | Viscosity | Injection | Delivery | |
| No. | Concentration | (mpa · s) | speed | speed | Phenomenon |

Imaging agent:CO₂:ethanol embolic agent
(containing no imaging agent):CO₂ = 10 μl:20 μl:30 μl:20 μl

| | | | | | | |
|---|---|---|---|---|---|---|
| 1 | 5% | 4.66 | 0.1 | ml/min | 1 ml/s | The injection speed |
| | | | | | | is too slow |
| | | | 20 | μl/s | 0.5 ml/s | Normal intermittent |
| | | | | | | injection |
| 2 | 10% | 7.64 | 20 | μl/s | 0.5 ml/s | Normal intermittent |
| | | | | | | injection |
| 3 | 15% | 12.76 | 20 | μl/s | 0.5 ml/s | Earlier intermittent |
| | | | | | | injection, and later |
| | | | | | | mixing together |

Imaging agent:CO2:ethanol embolic agent:CO2 = 10 μl:40 μl:30 μl:40 μl

| | | | | | | |
|---|---|---|---|---|---|---|
| 4 | 15% | 12.76 | 20 | μl/s | 0.5 ml/s | Mixing |
| | | | 10 | μl/s | 0.5 ml/s | Not mixing |
| 5 | 20% | 27.8 | 10 | μl/s | 0.5 ml/s | Later mixing |
| | | | 5 | μl/s | 0.5 ml/s | Not mixing the gas |
| | | | | | | being compressed |
| | | | | | | and shortened |

It is found from the above experiments that in the process of injection of the ethanol embolic agent prepared with the ethanol embolic agent, the imaging agent (iodophor), and CO₂, to ensure that the gas and the liquid components are not mixed, the injection speed decreases with increasing concentration of ethanol embolic agent. For example, for 5% PVP ethanol embolic agent, the maximum injection speed is 20 μl/s; and for 10% PVP ethanol embolic agent, the maximum injection speed is 20 μl/s.

It is to be noted that when an ethanol embolic agent containing an imaging agent (such as the ethanol embolic agent prepared with Povidone iodine) is used instead of a separate imaging agent, the injection performance is consistent with that in Table 8. For example, when the ethanol embolic agent containing imaging agent: CO2=30 μl:20 μl, the performance is the same as that of the 1st to 3rd groups of experiments in Table 8.

In the pharmaceutical composition containing ethanol provided in the present invention, Povidone is used as a thickener to adjust the viscosity, increase the concentration of ethanol in blood, and play a role in sustained release. Moreover, the thickener itself can be excreted, and will not form a solid in the blood vessels or tissues. The thickener increases the viscosity of the pharmaceutical composition and allows for the even distribution of the imaging agent. However, it can be understood that hydroxypropyl cellulose or ethyl cellulose can also be used as a thickener. Hydroxypropyl cellulose is preferably highly substituted hydroxypropyl cellulose.

In summary, the pharmaceutical composition containing ethanol provided in the present invention has the following notable advantages:

Compared with the technical solution using Onyx as an embolic agent, \textcircled{1} the pharmaceutical composition containing ethanol can not only effect occlusion, but also destroy cells; \textcircled{2} the cost is low, and no toxic or side effects are present; \textcircled{3} because the viscosity of the embolic agent is adjusted with Povidone, the pharmaceutical composition containing ethanol will not adhere to the tube, and be suitable for use with various kinds of microcatheters or puncture needles; \textcircled{4} the pharmaceutical composition containing ethanol has fast sclerosing ability, and can quickly form an embolism at fixed points in blood vessels; \textcircled{5} because ethanol is an organic solvent and a variety of imaging agents are soluble in it, an appropriate imaging agent can be added to improve the imaging ability of the embolic agent when imaged with X-ray or B-ultrasound; \textcircled{1} the pharmaceutical composition containing ethanol is non-corrosive to the catheter or the needle passage, so a common catheter (not a special catheter) or puncture needle can be used; \textcircled{7} due to the introduction of the microsphere technology, the drug-carrying performance of the embolic agent can be further improved, so as to improve the therapeutic effect; \textcircled{8} the pharmaceutical composition containing ethanol has no toxic and side effects, and the operation requirement is relatively low (low toxic DMSO needs to be injected before Onyx injection, and the injection speed of Onyx is slow); and \textcircled{9} the anti-inflammatory and bactericidal effects are used, to avoid infection after embolization.

Compared with the technical solution where anhydrous ethanol (including anhydrous ethanol and gelatin sponge) is directly used as an embolic agent, \textcircled{1} the pharmaceutical composition containing ethanol will not be diluted by the blood flow even in blood vessels with high blood flow, and the dosage can be accurately controlled; \textcircled{2} forward- or back-flow improper embolization is unlikely to be caused, to improve the therapeutic effect; \textcircled{3} the contact time between ethanol and vascular endothelial cells is prolonged, to avoid the formation of new endothelial cells; \textcircled{4} an imaging agent is contained, to make the embolism visible; and \textcircled{5} the anti-inflammatory and bactericidal effects are used, to avoid infection after embolization.

Therefore, the embolic agent provided in the present invention has the advantages of good operability, high embolization efficiency, high-definition imaging, large drug loading capacity and low cost. The ethanol-containing composition provided in the present invention can form a series of products with different CT values (the CT value is usually adjusted by adjusting iodine content), viscosity values (the viscosity is adjusted by adjusting the Povidone content) and ethanol contents or concentrations (by adjusting the ethanol content). Different products for different pathological blood vessels or tissues can be selected by the physician according to the clinical needs. For example, in soft tissues, such as liver, lung, breast and other soft tissue areas having low density, an ethanol embolic agent with relatively low iodine content (low CT value) provided in the present invention can be used; In the skull and bone area having high density, an ethanol embolic agent with high iodine content (high CT value) provided in the present invention is used to improve the diagnostic accuracy. For example, a pharmaceutical composition containing ethanol with high viscosity provided in the present invention is used in blood vessels with high blood flow rate; and a pharmaceutical composition containing ethanol with low viscosity provided in the present invention is used in blood vessels or tissues with slow blood flow rate. For example, an ethanol embolic agent with low ethanol content or concentration provided in the present invention is used in the veins; an ethanol embolic agent with high ethanol content or concentration provided in the present invention is used in the arteries; a sclerosing agent containing ethanol with low viscosity provided in the present invention is used in tissues for chemical ablation (such as liver tissue of patients with liver cancer); and a sclerosing agent containing ethanol provided in the present invention is used in mucosal surface tissues (such as lung nodules) in human lumens.

The ethanol embolic agent provided in the present invention can be formed into different preparations in a serial mode of solution-gas-solution-gas. For example, it can be stored in a microcatheter with a different liquid isolation mode such as first solution-gas-second solution-gas-first solution-gas-second solution. During use, the liquid in the microcatheter is injected into the blood vessel by an air pump, and mixed in the blood, to form an ethanol embolic agent or sclerosing agent. Alternatively, similar to Onyx, it is filled in a glass vial, drawn out with a needle, and then injected into blood vessels or tissues.

The pharmaceutical composition containing ethanol provided in the present invention has various applications. For example, it can be used in the treatment of angiogenesis, such as liver cancer, hepatic hemangioma and other liver diseases; bronchial artery embolization, and cerebral arteriovenous malformation (AVM) embolization; occlusion of the fallopian tube to achieve contraception or prevent the sactosalpinx from entering the uterine cavity to affect the embryo implantation; and embolization and sclerosis of aneurysms and venous tumors. The ethanol embolic agent provided in the present invention can be used not only in vascular embolic agents, but also for embolization in fistulas (for example, by injecting into hepatic arteriovenous fistula, and alveolopleural fistula) and crysts (for example, by injecting into cystonephrosis, and ovarian chocolate cyst). It will not damage the tissues outside the focus (for example, in pancreatic pseudocyst communicating with the main pancreatic duct, the main pancreatic duct will be damaged if anhydrous alcohol is injected directly), and has the functions of destroying the inner layer of the cyst wall, sterilization, and embolization. It can be seen that the embolic agent provided in the present invention can replace Onyx embolic agent, and has better effect.

The pharmaceutical composition containing ethanol provided in the present invention can be used for treating tumors, by injecting into tumor tissues, to dehydrate, denature coagulate and kill the cells, and is thus suitable for chemical ablation. For example, it is injected into the bladder tumor tissue under a cystoscope, and into the tumor tissue of lung cancer under a fiberoptic bronchoscope, to dehydrate and coagulate the tumor tissue. Alternatively, the pharmaceutical composition containing ethanol provided in the present invention is injected into coronary artery septum to treat hypertrophic cardiomyopathy.

In addition, the pharmaceutical composition containing ethanol provided in the present invention can be used for treating varicose veins of lower limbs and the like. Since the pharmaceutical composition containing ethanol provided in the present invention is in a liquid state after being injected into the blood vessel through a microcatheter and no solid material is left in the blood vessel, so the morphology of the blood vessel will return to the natural state (keeping the blood vessel hollow), and the appearance of the patient's skin will not be affected. This is different from Onyx, because when Onyx is used as an embolic agent, the solid embolic material will fill the blood vessels after the operation (making the blood vessels solid), and the blood vessels filled with the solid embolic material will protrude out of the normal skin, to form an earthworm shape, affecting the appearance.

The pharmaceutical composition containing ethanol provided in the present invention can be used for treating chronic obstructive pulmonary disease (COPD). The characteristic pathological change of COPD is limited airflow, which is a result of the joint action of small airway lesion (bronchiolitis obliterans) and lung parenchyma destruction (emphysema). Smoking can induce the inflammation of hair-like cells (cilia) in the airway. The activated inflammatory cells release a variety of mediators, which can destroy the lung structure and/or cause the inflammatory reaction of neutrophils. After injection into bronchioles and alveoli, the pharmaceutical composition containing ethanol (contrast agent iodine) can destroy epithelial and mucinous cells on the bronchioles and alveoli, and block the small airways and alveolar pores, to reduce the local lung volume, and achieve the effect of local lung volume reduction. The composition also has the functions of sterilization and bacteriostasis, and as an anti-inflammatory agent, can effectively prevent the complicated inflammatory reaction in the occlusion process.

The pharmaceutical composition containing ethanol provided in the present invention can be used for treating hyperparathyroidism. When percutaneously injected into parathyroid gland under B-ultrasound or X-ray, an appropriate amount of the pharmaceutical composition containing ethanol provided in the present invention can reduce the volume of parathyroid gland and the level of parathyroid hormone.

The pharmaceutical composition containing ethanol provided in the present invention can also be used for relieving pain of ganglia and nerve roots. For example, when injected into the semilunar ganglion, the pharmaceutical composition containing ethanol provided in the present invention can alleviate or eliminate trigeminal neuralgia. When injected into the celiac plexus, the pharmaceutical composition containing ethanol provided in the present invention can relieve the cancer pain of the upper abdomen (such as unresectable pancreatic cancer).

The pharmaceutical composition containing ethanol provided in the present invention can also be used for local hemostasis. For example, in suprapubic intravesical prostatectomy, the major difficulty is to stop bleeding. In the operation, the injection of the pharmaceutical composition containing ethanol can stop bleeding quickly, and the pharmaceutical composition can be distributed in a large area, so that enough ethanol can infiltrate the whole gland to stop bleeding effectively.

The gelatinous pharmaceutical composition containing ethanol provided in the present invention can also be used for treating hydronephrosis and renal cyst. An appropriate amount of the pharmaceutical composition containing ethanol provided in the present invention is injected by percutaneous puncture under the guidance of B-ultrasound or X-ray, and observed with the aid of the imaging agent contained. After a predetermined time (for example, 10 min to 1 hr), if the imaging agent is observed to be obviously reduced or disappeared in the image, hydronephrosis is determined; and if the imaging agent does not disappear or decrease obviously, renal cyst is determined. Because of the agglutination reaction between the protein and ethanol, the ethanol in the composition provided in the present invention is consumed. Therefore, the gelatinous ethanol-containing composition provided in the present invention is no longer gelatinous, so it cannot be observed under B-ultrasound or X-ray.

In addition, because the pharmaceutical composition containing ethanol provided in the present invention remains gelatinous/pasty and does not solidify, in an application scenario using a puncture needle, injection of the gelatinous/pasty embolic agent provided in the present invention in the process of withdrawing the needle can close the needle passage, to prevent the transfer of implantation of the needle passage, and prevent bleeding or infection in the needle passage.

The pharmaceutical composition containing ethanol provided in the present invention has high imagining ability; sufficient fluidity (injectable by a microcatheter having the smallest aperture); a certain inflammatory reaction (causing the embolized blood vessels to be permanently occluded); and no toxic or side effects on normal tissues, will not solidify in blood, so there is no difficulty in withdrawing the tube; and can achieve permanent embolization by means of its destructive ability to endothelial cells. Therefore, it can be widely used as an embolic agent, sclerosing agent or drug delivery carrier.

It is to be noted that although some of the above applications require direct injection of anhydrous ethanol, the pharmaceutical composition containing ethanol provided in the present invention can be anhydrous ethanol, a composition with lower concentration of ethanol, because the thickener can increase the concentration of ethanol and increase the acting time of ethanol on tumor tissues.

The pharmaceutical composition containing ethanol provided in the present invention has high imaging ability, sufficient fluidity, and no toxic or side effects on normal tissues; will not solidify in blood, and can achieve permanent embolization by means of its destructive ability to endothelial cells; and can affect the sclerosis of cells in tissues or erosion or sclerosis of mucosal surface tissues in human lumens.

The technical solution of the present invention has been described in detail above. For those of ordinary skill in the art, any obvious changes made to the present invention without deviating from its essence will constitute an infringement of the patent right of the present invention, and should bear corresponding legal responsibilities.

1. A pharmaceutical composition containing ethanol, for use in animals, comprising: ethanol with a concentration of not less than 50% and a liquid ethanol thickener.

2. The pharmaceutical composition containing ethanol according to claim 1, wherein ethanol is added in an amount of 10 to 60 g/ml; and the liquid ethanol thickener is selected from Povidone, low-molecular glycerol, a syrup, a macromolecular hydrophilic carbohydrate polymer or hydrophilic non-carbohydrate polymer.

3. The pharmaceutical composition containing ethanol according to claim 1, wherein the amount of the liquid ethanol thickener added is 15 to 45 g/ml.

4. The pharmaceutical composition containing ethanol according to claim 1, further comprising an imaging agent.

5. The pharmaceutical composition containing ethanol according to claim 1, further comprising a calcium acetate solution.

6. The pharmaceutical composition containing ethanol according to claim 1, further comprising an alkaline salt, selected from sodium bicarbonate, calcium carbonate, sodium acetate, sodium phosphate, sodium hypochlorite, sodium sulfite, ammonium bicarbonate, calcium hypochlorite, sodium metaaluminate or sodium carbonate; and the pH value of the alkaline salt powder is 7-10.

7. An embolic agent or a sclerosing agent for chemical ablation, comprising the pharmaceutical composition containing ethanol according to claim 1.

8. A gas-liquid preparation comprising the pharmaceutical composition containing ethanol according to claim 1, comprising a plurality of sections of the pharmaceutical composition containing ethanol separated by a gas.

9-10. (canceled)

11. Use of the pharmaceutical composition containing ethanol according to claim 1 as a sclerosing agent for mucosal surface tissues in human lumens.

12. Use of the pharmaceutical composition containing ethanol according to claim 1 as an anti-inflammatory agent.

13. Use of the pharmaceutical composition containing ethanol according to claim 1 in the preparation of drugs for treating hyperparathyroidism.

14. Use of the pharmaceutical composition containing ethanol according to claim 1 in the preparation of analgesics for ganglia and nerve roots.

15. Use of the pharmaceutical composition containing ethanol according to claim 1 in the preparation of drugs for local blood occlusion or occluding the needle passage is provided.
